(19) United States
(12) Patent Application Publication
(10) Pub. No.: US 2019/0231561 A1
(43) Pub. Date: Aug. 1, 2019

(54) PROSTHETIC FOOT, AND PROSTHESIS FOR A LOWER EXTREMITY

(71) Applicant: Andreas RADSPIELER, Neubeuern (DE)

(72) Inventor: Andreas RADSPIELER, Neubeuern (DE)

(21) Appl. No.: 16/316,920

(22) PCT Filed: Jul. 20, 2017
(86) PCT No.: PCT/EP2017/068338

§ 371 Date: Jan. 10, 2019

(30) Foreign Application Priority Data
Jul. 20, 2016 (DE) 10 2016 113 391.4
Oct. 5, 2016 (DE) 10 2016 118 834.4
Mar. 13, 2017 (DE) 10 2017 105 244.5

(51) Int. Cl. A61F 2/66

(52) U.S. Cl. CPC A61F 2/66; A61F 2002/6657; A61F 2002/5038; A61F 2002/6685; A61F 2002/6614; A61F 2002/665

(57) ABSTRACT

A prosthesis foot is provided with a structural component with proximal connecting device for fixing the prosthesis foot to a lower leg tube, lower leg shaft or a prosthesis knee joint, wherein the proximal connecting device is designed as a joint or comprises as at least one joint.

The present invention relates to a prosthesis shaft and a prosthesis for the lower extremity.

Leg amputees may regain mobility using leg prostheses. Modern leg prostheses include various modules (prosthesis shaft, knee, lower leg and foot modules), which may be combined to meet the various needs of the prosthesis wearer (hereinafter referred to as wearer or patient) in terms of fundamental mobility, sport activities and aesthetic perceptions.

In amputations, it is advantageous to obtain a long stump, see e.g. Pyrogoff, Syme's amputation and hindfoot-restraining amputation lines or very far distally amputated tibia amputations.

However, this stump length which is advantageous for the patient presents a challenge for the prosthetic care.

Because of the comparatively long stump conditions, often only 0-10 mm “mounting space” remain available to mount a prosthetic foot on the prosthesis shaft. Since these feet must have very low mounting heights, their dynamics of rolling behavior suffer thereunder. Such feet are often very rigid and have little dynamics due to the low bending possibilities.

It may be an object of the present invention, to propose further apparatuses for the prosthetic care of the lower extremity.

The object of the present invention may be achieved by the prosthetic foot having the features of claim 1. It may further be achieved by the prosthesis having the features of claim 13.

Thus, a prosthetic foot with a structural component having a proximal connecting device for fastening the prosthetic foot e.g. to a lower leg tube, lower leg shaft or prosthetic knee joint is proposed, wherein the proximal connecting device is designed as—preferably exactly one—joint or comprises exactly one joint or at least one joint.

The prosthesis according to the present invention comprises a prosthetic foot according to the present invention. It further optionally comprises a lower leg tube, a lower leg shaft and/or a prosthetic knee joint.

Embodiments according to the present invention may comprise one or several of the following features in any combination, unless the person skilled in the art recognizes a particular combination as being clearly technically impossible. Also the subject-matters of the dependent claims indicate embodiments according to the present invention.

In all of the following statements, the use of the expression “may be” or “may have” and so on, is to be understood synonymously with “preferably is” or “preferably has,” and so on, respectively, and is intended to illustrate embodiments according to the invention.

Whenever numerical words are mentioned herein, the person skilled in the art shall recognize or understand them as indications of numerical lower limits. Hence, unless it leads to a contradiction evident for the person skilled in the art, the person skilled in the art shall comprehend for example “one” as encompassing “at least one”. This understanding is also equally encompassed by the present invention as the interpretation that a numerical word, for example, “one” may alternatively mean “exactly one”, wherever this is evidently technically possible in the view of the person skilled in the art. Both of these understandings are encompassed by the present invention and apply herein to all used numerical words.

Whenever spatial information or references such as “top”, “bottom”, “upper”, “lower”, are mentioned herein, the person skilled in the art understands them, when in doubt, as spatial information with reference to the orientation or alignment as seen in the accompanying figures.

In several exemplary embodiments according to the present invention, the prosthetic foot comprises a structural component with the proximal connecting device for fastening the prosthetic foot to a lower leg tube, lower leg shaft or prosthesis knee joint. In this, the proximal connecting device—preferably in its entirety—is designed as—preferably exactly one—joint (in particular rotating joint) or comprises at least one joint, wherein the prosthetic foot comprises at least one elastic element or is connected thereto. Further, the elastic element is, in a first area thereof, connected, in particular releasably, to the structural component. In addition, the elastic element comprises a second area for contacting the lower leg tube, lower leg shaft or prosthesis knee joint, in particular at a side, front side or rear side of the lower leg tube, lower leg shaft or prosthesis knee joint.

In several exemplary embodiments according to the present invention, the proximal connecting device is designed as —preferably exactly one—joint or comprises at least one joint on which a spring effect or restoring effect may be applied without using a spring or elastic element.

In several exemplary embodiments according to the present invention, the lower leg tube, the lower leg shaft or the prosthetic knee joint is connected to the structural component by only a single connecting device, e.g. only one joint.

In several exemplary embodiments according to the present invention, the lower leg tube, the lower leg shaft or the prosthetic knee joint comprises no hydraulic shock absorber.

In several exemplary embodiments according to the present invention, no spring is part of the lower leg tube, lower leg shaft or prosthetic knee joint.

In several exemplary embodiments according to the present invention, the lower leg tube, the lower leg shaft or the prosthetic knee joint is connected to, or contacts, the structural component by only one single elastic element.

In several exemplary embodiments according to the present invention, the lower leg tube, the lower leg shaft or the prosthetic knee joint is not connected to, nor contacts, the structural component by an elastic element which is designed as spiral spring or comprises a spiral spring.

In several exemplary embodiments according to the present invention, the joint is monocentric or polycentric.

In several exemplary embodiments according to the present invention, the joint is monoaxial or polyaxial.

In several exemplary embodiments according to the present invention, the joint has exactly one joint axis.

In several exemplary embodiments according to the present invention, the joint axis of the joint is not diagonal or oblique relative to the longitudinal axis of the structural component (e.g. of the foot sole), alternatively, it is not only diagonal relative to the longitudinal axis of the structural component.

In several exemplary embodiments according to the present invention, the structural component is a foot sole, comprises same or is connected thereto.

In several exemplary embodiments according to the present invention, the foot sole is a structure, having a flat upper surface and/or underside e.g. a plate.

In several exemplary embodiments according to the present invention, the elastic element is one or exactly one leaf spring or one or exactly one bending spring, comprises same or is connected thereto.

In several exemplary embodiments according to the present invention, the elastic element extends, at least in sections thereof, along the lower leg shaft, preferably in a distal-proximal direction, preferably along an exterior of the lower leg shaft, preferably without a fixed connection to the lower leg shaft, preferably guided laterally, preferably not screwed, preferably not firmly bonded, preferably not form-fit connected, preferably not force-fit connected.

In several exemplary embodiments according to the present invention, the elastic element lies dorsally or ventrally contacting the lower leg tube, lower leg shaft or prosthesis knee joint, preferably not medially and/or not laterally.

In several exemplary embodiments according to the present invention, an adjustable element is provided for releasably positioning a section of the elastic element to the lower leg tube, lower leg shaft or prosthesis joint.

In several exemplary embodiments according to the present invention, a guiding element, e.g. a sliding element, is provided for guiding—in particular in a sliding manner—a section of the elastic element relative to the lower leg tube, lower leg shaft or prosthesis knee joint, in particular in a distal-proximal direction or a downward-upward direction, and/or in a left-right direction or a lateral-medial direction.

In several exemplary embodiments according to the present invention, the slide element comprises no screw, in particular no screw extending through the elastic element.

In several exemplary embodiments according to the present invention, the elastic element comprises no transverse or longitudinal slot in the area of the second area.

In several exemplary embodiments according to the present invention, no screw in inserted through the elastic element in the area of the second area.

In several exemplary embodiments according to the present invention, statements made herein concerning the adjustable element relate as well to the slide element, and vice versa.

In several exemplary embodiments according to the present invention, the elastic element does is not in contact with the lower leg tube, lower leg shaft or prosthesis knee joint between the first area and the second area.

In several exemplary embodiments according to the present invention, the second area is optionally arranged to be shiftable relative to the lower leg tube.

In several exemplary embodiments according to the present invention, the second area lies optionally in an end area of the elastic element, e.g. the bending spring.

In several exemplary embodiments according to the present invention, an intermediate element lies between the second area and the lower leg shaft. The intermediate element may be part of the elastic element, e.g. of the bending spring, or part of the lower leg shaft. The intermediate element may be arranged to be shiftable relative to the second area, shiftable relative to the lower leg shaft or shiftable relative to both.

The intermediate element may be designed as an adapter between the geometries of the second area on the one hand and the lower leg shaft on the other.

An adjusting device may be provided to secure or fix the intermediate element in a predetermined or desired height of the lower leg shaft or of the elastic element, (e.g. of the spring, in particular of the leaf spring or of the bending spring) to the lower leg shaft or to the elastic element. Therethrough, the spring effect may be influenced.

In several exemplary embodiments according to the present invention, the prosthetic foot and/or the elastic element comprises an adjusting device for changing the height of the second area above the foot sole or for changing the distance between the first area and the second area.

In several exemplary embodiments according to the present invention, the spring, in particular the leaf spring or the bending spring, preferably between at least its first area and its second area, is, in the unloaded condition and/or in the mounted state when the prosthetic foot is not loaded by the patient, a straight and/or a flat spring and/or a spring which is not curved along one or all its longitudinal axes.

In several exemplary embodiments according to the present invention, the length of the spring is at least 20 cm, 25 cm, 30 cm, 35 cm, 40 cm or longer. Therewith there is ensured that an optimum spring effect may be achieved also during high activity of the prosthesis wearer.

In several exemplary embodiments according to the present invention, the spring has a constant thickness.

In several exemplary embodiments according to the present invention, the spring has a constant width.

In several exemplary embodiments according to the present invention, the width of the spring is at least 3 cm, preferably at least 4 cm, more preferably at least 5 cm, very most preferably 6 cm.

The foregoing information concerning the possible geometry of the spring allow an optimal force application by the spring with durability at the same time and comparatively low spring weight and simple and inexpensive manufacturability.

In several exemplary embodiments according to the present invention, the second area of the elastic element e.g. of the spring, contacts the prosthesis, e.g. the lower leg prosthesis or the prosthetic knee joint, in an upper area or upper edge area. Alternatively, it is optionally connected to a middle area (with regard to the length).

In several exemplary embodiments according to the present invention, the longitudinal axis of the spring is at an angle of preferably 70° to 110° relative to the main extension plane of the foot sole, preferably between 80° and 100°, most preferably between 85° and 95°. Due to the fact that the spring in these embodiments protrudes more or less perpendicularly from the foot sole (e.g. from the bottom side thereof and/or the upper side thereof), a progressive spring effect with restoring effect is ensured with an increasing bending e.g. of the knee joint, which may contribute to the natural gait pattern of the prosthesis wearer.

In several exemplary embodiments according to the present invention, the elastic element applies force on the lower leg tube, the lower leg shaft or the prosthesis knee joint only in the second area.

In several exemplary embodiments according to the present invention, the elastic element extends proximally.

In several exemplary embodiments according to the present invention, the elasticity of the foot sole (in particular within said sole itself or along its longitudinal extension) is not influenced by an elastic element and/or by a joint and/or has neither an elastic element nor a joint serving for this purpose.

In several exemplary embodiments according to the present invention, the structural component comprises no heel-side spring damper system which, at a heel strike, becomes compressed and supports itself e.g. on a sole-side guiding element.

In several exemplary embodiments according to the present invention, the structural component or its foot sole is not designed as a spring, in particular not as a bending spring or a leaf spring, and/or does not comprises such same.

In several exemplary embodiments according to the present invention, the prosthetic foot is connected to a lower leg tube, lower leg shaft or prosthetic knee joint.

In several exemplary embodiments according to the present invention, the first area is firmly fixed or non-releasably connected to the structural component.

In several exemplary embodiments according to the present invention, the first area is releasably connected to the structural component.

In several exemplary embodiments according to the present invention, the first area is directly or indirectly screwed to the structural component.

In several exemplary embodiments according to the present invention, the first area is connected to the structural component under a predetermined angle to thereto.

In several exemplary embodiments according to the present invention, the first area is shiftably connected to the structural element along the latter. Therefore, the first area—for example arranged on a slide—may be arranged on one or several guiding elements (e.g. guiding rails) in a shiftable manner. It may be connected to the guiding elements by one, two or several shiftable guiding devices. In this, some of these guiding devices may be arranged in front of (ventrally) and others behind (dorsally) the elastic element.

In several exemplary embodiments according to the present invention, the proximal connecting device is shiftably connected to the structural component. Therefore, the proximal connecting device—for example arranged on a slide—may be arranged on guiding elements (e.g. guiding rails) in a shiftable manner. The guiding elements may be the aforementioned elements on which the elastic element is already shiftably arranged. Hence, the distance between the proximal connecting device and the elastic element, in particular its first area, may be easily varied or modified. Alternatively, not only one, rather several guiding elements are provided. For example, one guiding element for the elastic element, another for the proximal connecting device.

In several exemplary embodiments according to the present invention, the structural component is not connected via the proximal connecting device to a fork or fork-shaped section of the lower leg tube, lower leg shaft or prosthesis knee joint.

In several exemplary embodiments according to the present invention, the structural component comprises a device for adjusting the angle, under which the first area is connected to the structural component. In other embodiments according to the present invention, there is no such adjusting device provided.

In several exemplary embodiments according to the present invention, the first area is connected to the structural component by a connection like latching, snap-in, clamping or another connection, e.g. by a device which allows a tilting of the first area (e.g. in a lateral-medial direction) as well as a shifting thereof (e.g. in a dorsal-ventral direction).

In several exemplary embodiments according to the present invention, the proximal connecting device is designed as at least one joint (or comprises such) which is connected to an extension, e.g. in the form of a tube or lower leg tube.

In several exemplary embodiments according to the present invention, the joint of the connecting device is connected to the structural component by a shifting device.

The shifting device may allow a shifting in the ventral-dorsal direction. It may optionally allow (additionally or alternatively) an adjustability in a lateral-medial direction.

In several exemplary embodiments according to the present invention, the first area is connected to the structural component by a shifting device.

This shifting device may allow a shifting in the ventral-dorsal direction. It may optionally allow (additionally or alternatively) an adjustability and/or movability in a lateral-medial direction.

In several exemplary embodiments according to the present invention, the shifting devices may be arranged in order to influence, e.g. increase, the spring effect of the elastic element, e.g. the bending spring.

The shifting devices may be adjusting devices for the elastic element.

In several exemplary embodiments according to the present invention, the prosthesis or the prosthetic foot comprises a connector which at least with one section thereof is arranged between the lower leg shaft and the second area.

In several exemplary embodiments according to the present invention, the connector is loosely arranged between the lower leg shaft and the second area.

In several exemplary embodiments according to the present invention, the connector is releasably or non-releasably connected to the lower leg shaft and/or the second area in a form-fit connection.

In several exemplary embodiments according to the present invention, the connector may be designed a rod or a tube, wherein the connector extends preferably from the structural component to the lower leg shaft.

In several exemplary embodiments according to the present invention, the connector is a connecting link between the prosthesis shaft, e.g. lower leg shaft, and the elastic element (e.g. bending spring) or comprises an intermediate element such as a connecting link.

In several exemplary embodiments according to the present invention, the connector or the connecting link is designed to effect a balance between the lower leg shaft and the elastic element, e.g. the bending spring, in a lateral-medial direction, e.g. for balancing a lateral shifting (e.g. in the medial-lateral direction).

In several exemplary embodiments according to the present invention, the connector and/or the intermediate element is designed to effect a balance between the curved surface of the lower leg shaft and the optionally straight surface of the bending spring.

In several exemplary embodiments according to the present invention, the optional connector is connected to the structural component, in a further section lying distally to the section e.g. to its foot sole.

In several exemplary embodiments according to the present invention, the further section is (optionally also) connected to the structural component by a shifting device.

In several exemplary embodiments according to the present invention, the further section is mounted free of any spring or restoring effect and/or is not provided with a spring.

In several exemplary embodiments according to the present invention, the prosthesis comprise the prosthetic foot and the lower leg shaft or consists thereof.

In several exemplary embodiments according to the present invention, all or several of the connections are shiftable to each other.

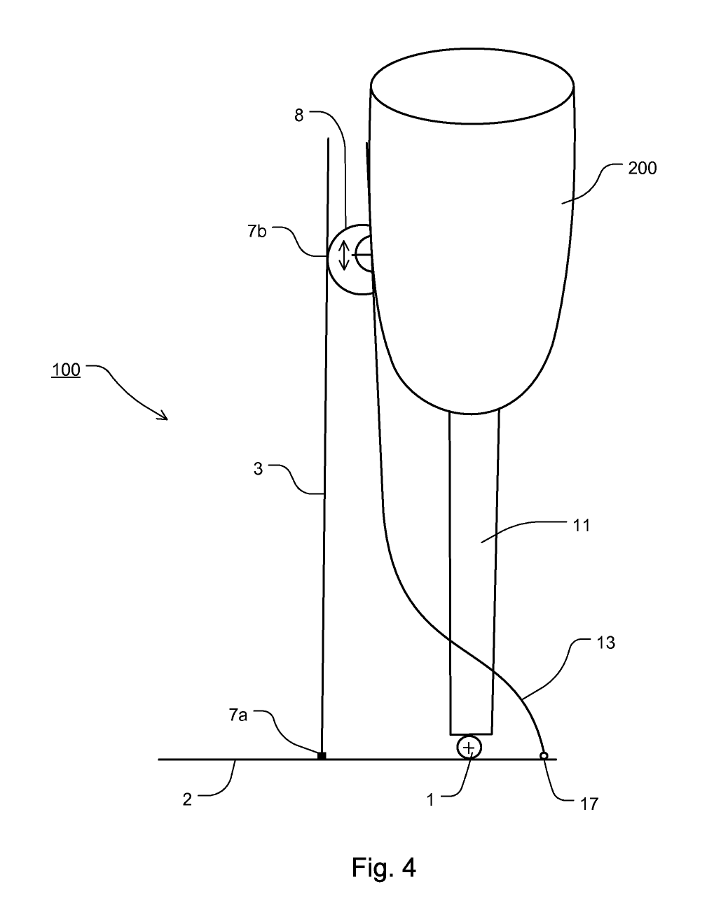

In several exemplary embodiments according to the present invention, the optional connector is connected to the structural component, e.g. to its foot sole, by the section which is ventral to the connecting device, while said section lies alternatively dorsally (see e.g. FIG. 4) to the connecting device.

In several exemplary embodiments according to the present invention, the connector, when viewed from the side, extends from ventral of the lower leg shaft in the area of the second section to the dorsal side of the lower leg shaft, where it is connected in the section to the foot sole.

In several exemplary embodiments according to the present invention, the intermediate element is designed as being a height-shiftable or height-adjustable section and/or is connected to the bending spring. Thus, the spring effect may be influenced.

In several exemplary embodiments according to the present invention, the intermediate element comprises at least one, optionally two, three, four or more curved surfaces or sections (herein each denoted in short: surface). The elastic element, e.g. the bending spring, may abut or rest on the curved surfaces or, depending on the load of the prosthetic foot by the wearer, contact the latter. One, several or all of the curved surfaces or sections may be formed convexly towards the bending spring. Thus, each of the curved surfaces may on the one hand support or guide (e.g. by its vertex) the bending spring and on the other hand the bending spring is not excessively limited in its movement, e.g. in an inclination movement and/or in a tilting movement.

In several exemplary embodiments according to the present invention, the first area is designed as a joint or comprises at least one joint. The joint may be monocentric or polycentric.

In several exemplary embodiments according to the present invention, the joint of the first area is monoaxial or polyaxial.

In several exemplary embodiments according to the present invention, the first area is a hinge or comprises a hinge.

In several exemplary embodiments according to the present invention, the first area is a saddle joint or comprises a saddle joint.

In several exemplary embodiments according to the present invention, the first area is a ball joint or comprises a ball joint.

In several exemplary embodiments according to the present invention, the elastic element is removable from the intermediate element. A removable elastic element may be referred to as releasable or separable with respect to the intermediate element.

In several exemplary embodiments according to the present invention, the elastic element and/or the foot sole is made of or comprises a plastic, a glass-fiber reinforced material, a carbon-fiber reinforced material, a composite material, a metallic material or combinations of these materials.

In several exemplary embodiments according to the present invention, the elastic element is fixed in or at or to the intermediate element. The fixing may be a releasable or a non-releasable connection, e.g. a material-fit or form-fit connection. The elastic element may be clamped or glued in the intermediate element or differently fixed or fastened there.

In several exemplary embodiments according to the present invention, the intermediate element is connected to the lower leg shaft by a further connecting device. The further connecting device may be a joint, e.g. a polyaxial and/or a polycentric joint and/or a hinge, a saddle joint or a ball joint or may comprise same. Optionally, the further connecting device comprises no joint effect.

The further connecting device may be connected to the lower leg shaft and/or to the intermediate element releasably or non-releasably.

In several exemplary embodiments according to the present invention, the structural component and/or the proximal connecting device comprises no spiral spring and is not in contact or engaged thereto, in particular not to more than one spiral spring.

Several or all of the embodiment according to the present invention may comprise one or several of the aforementioned or following advantages.

By using the apparatuses according to the present invention, the joint motion and the dynamic springing or resilience are technically decoupled. A significantly improved dynamic when the patients are walking is thus achieved. The dynamics may be adjusted separately from the general conditions that the joint connection provides.

The prosthetic foot construction suggested herein may also be advantageous, if after a knee exarticulation or a transfemoral amputation, an artificial knee joint with the necessary tube screwing only allows the use of a very flat prosthetic foot (knee-floor dimension)

The prosthetic foot according to the present invention may advantageously be used for both averagely long as well as for short treatments of lower leg stumps.

An advantage according to the present invention may furthermore be that the helpful adjustment possibility may be maintained by the screw connections of the components between foot, tube and lower leg shaft.

The height adjustability of the holding element e.g. in form of a holding ribbon enables or serves for selecting the spring length with a heel strike. Depending on the activity or safety requirements of the patient, one may change the length of the spring, if necessary, also freely, and preferably the patient him/herself without using tools, respectively.

Furthermore, regardless of the length of the stump, a large spring dynamic may be achieved. In this, there is a possibility to adjust the foot position or positioning, which is very similar to the foot positioning of the foot when using it physiologically. It is also possible to adjust abduction and adduction as well as dorsiflexion and plantar flexion even far distally. In this, the point of rotation of the adjustment advantageously does not shift undesirably far proximally.

In the case of long stump conditions, usually only 0-10 millimeters of the available “mounting space” are needed for mounting a prosthetic foot on the prosthesis shaft. Despite the possibly very low mounting height, the dynamics of the rolling behavior must not suffer therefrom.

The present invention will be hereinafter exemplarily explained with reference to the accompanying drawings. The following applies in the partly highly simplified figures:

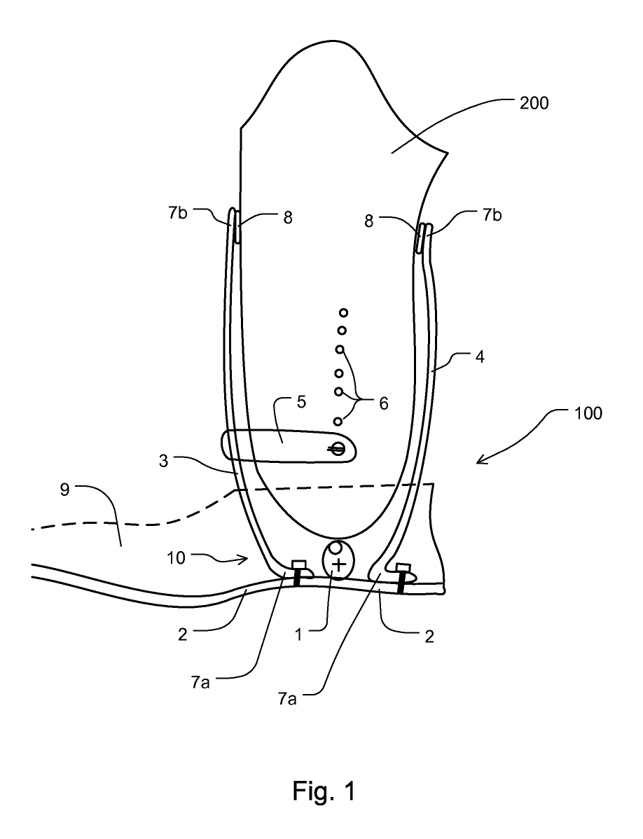

FIG. 1 shows a prosthesis according to the present invention with a prosthetic foot according to the present invention in a first embodiment in a longitudinal section view, exemplarily fastened to a lower leg shaft;

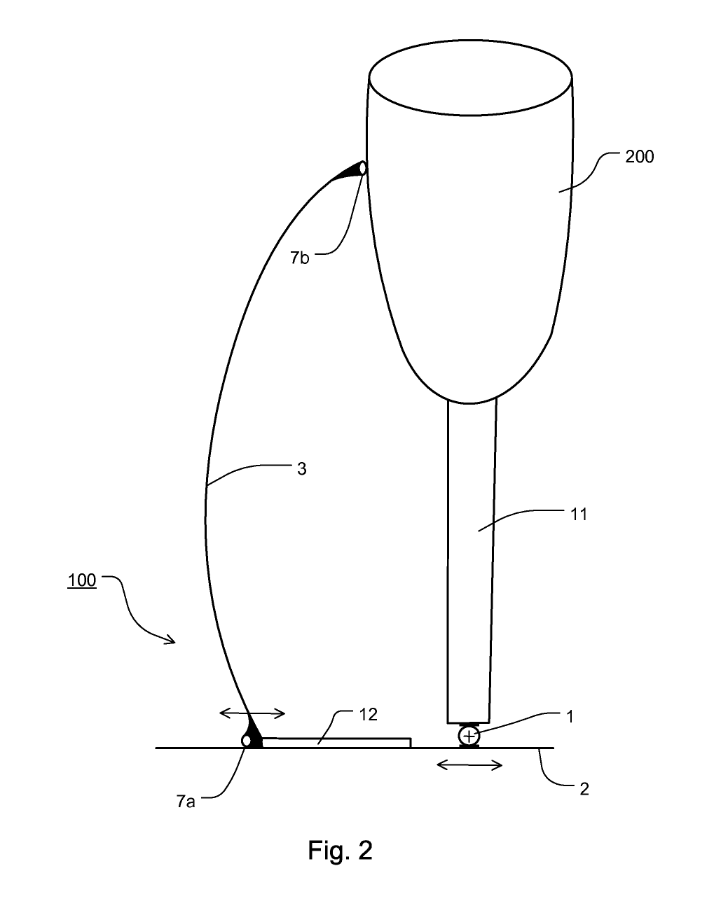

FIG. 2 shows a prosthesis according to the present invention with a prosthetic foot according to the present invention in a second embodiment in a lateral view, exemplarily fastened to a lower leg shaft;

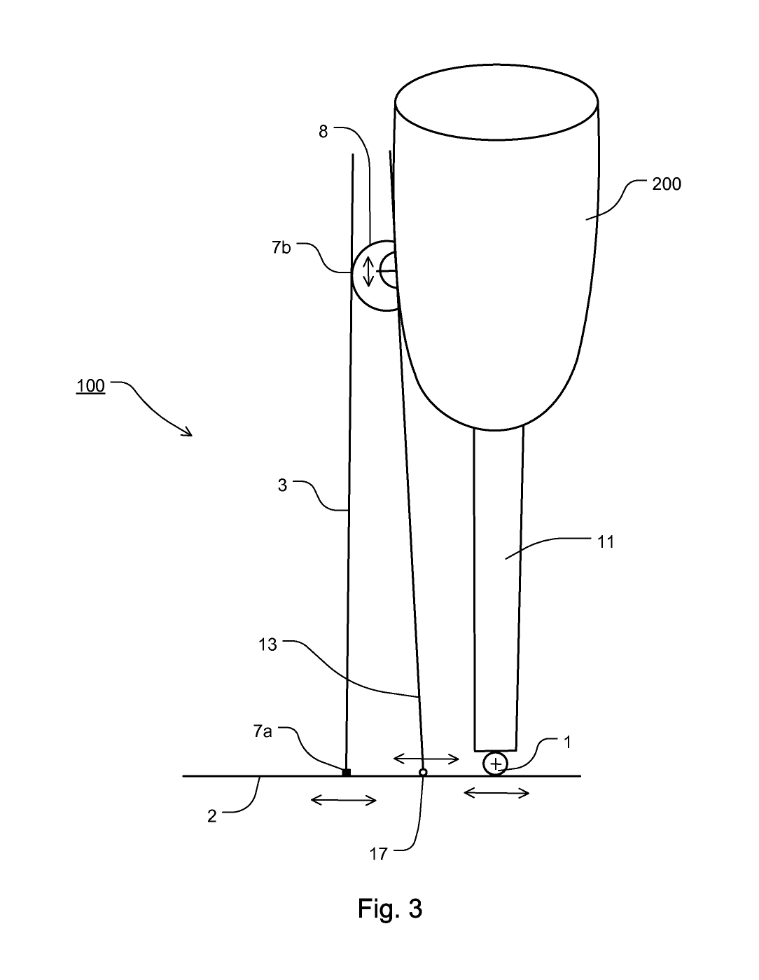

FIG. 3 shows a prosthesis according to the present invention with a prosthetic foot according to the present invention in a third embodiment in a lateral view, exemplarily fastened to a lower leg shaft;

FIG. 4 shows a prosthesis according to the present invention with a prosthetic foot according to the present invention in a fourth embodiment in a lateral view, exemplarily fastened to a lower leg shaft;

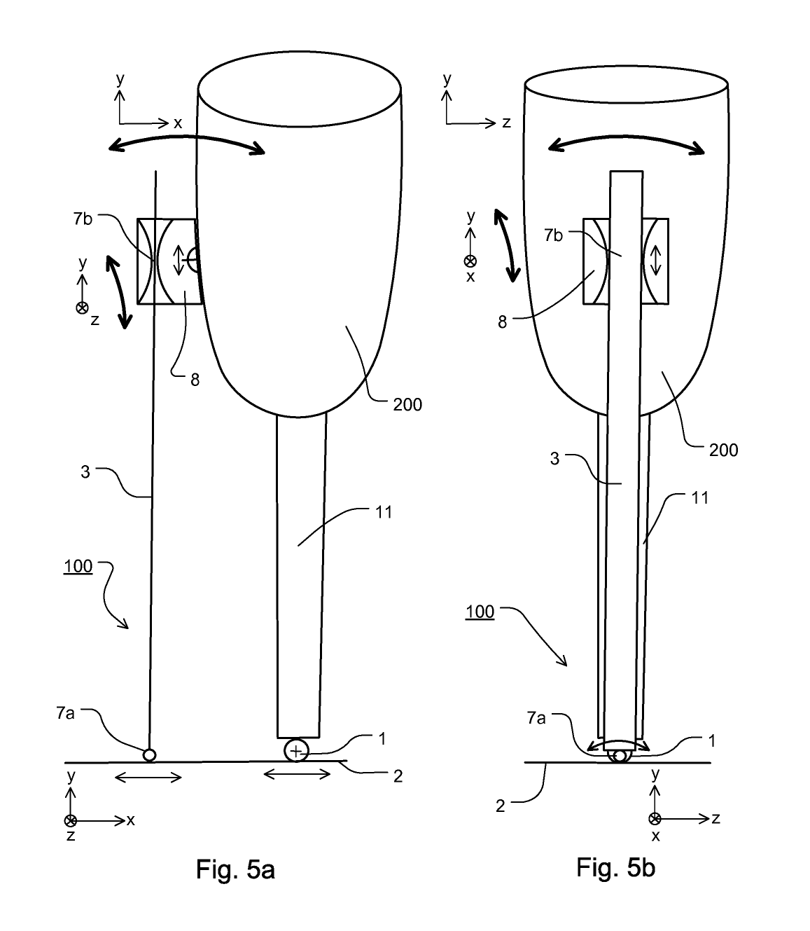

FIG. 5a,b show a prosthesis according to the present invention with a prosthetic foot according to the present invention in a fifth embodiment in a lateral view (FIG. 5a) and in a view from the front (FIG. 5b) twisted about 90 degrees for this purpose;

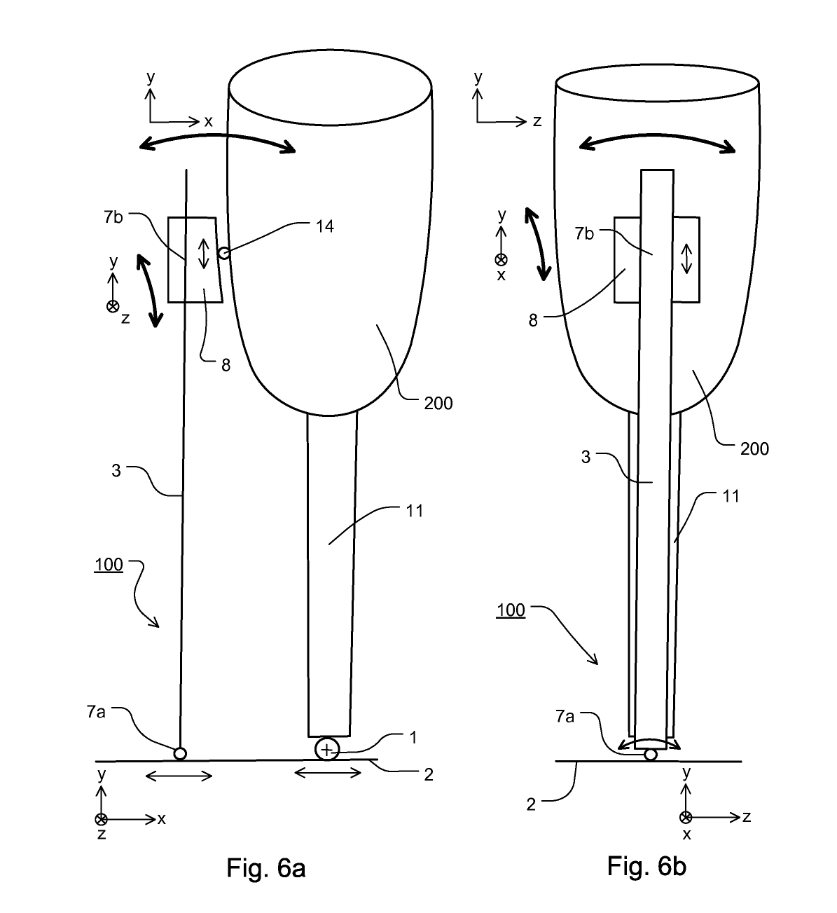

FIG. 6a,b show a prosthesis according to the present invention with a prosthetic foot according to the present invention in a sixth embodiment with a further intermediate element; and

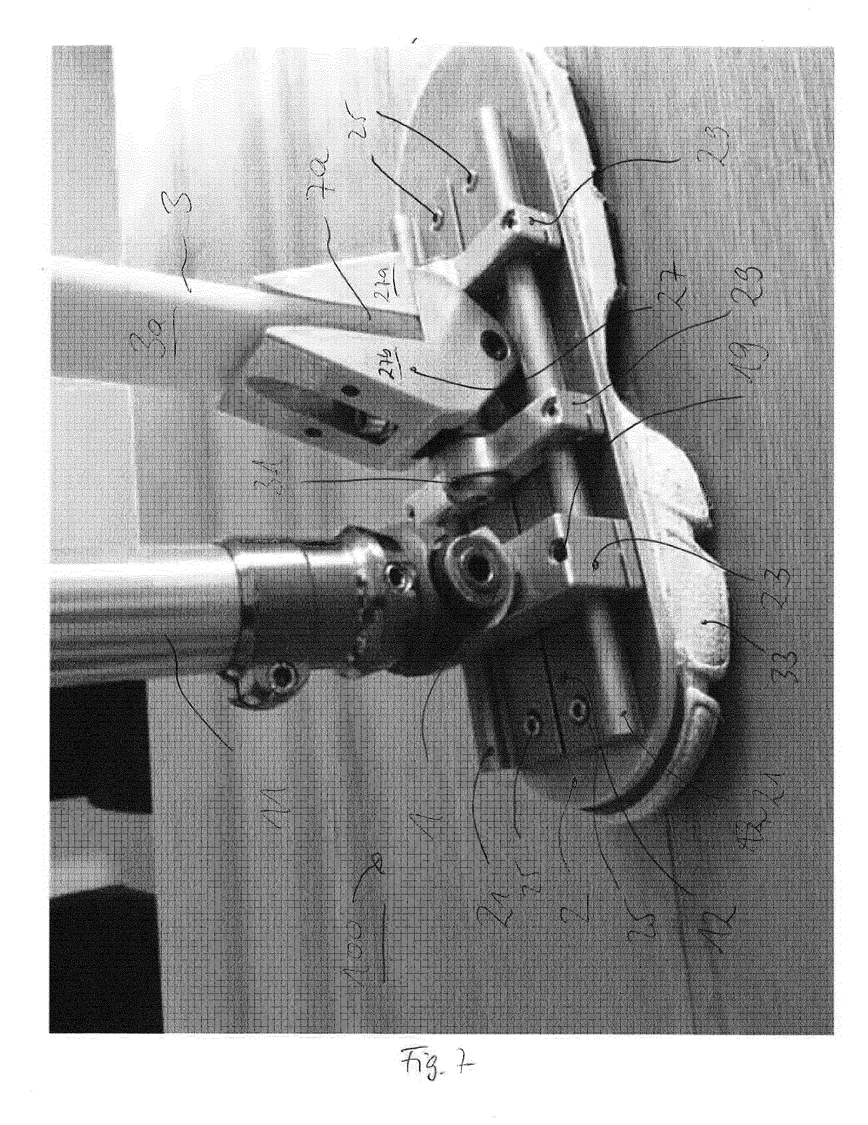

FIG. 7 shows a prosthetic foot according to the present invention in a further embodiment.

FIG. 1 shows in a purely exemplary embodiment, a prosthetic foot 100 according to the present invention in a first embodiment in a longitudinal section view, fastened to a lower leg shaft 200 (alternatively, to a lower leg tube, a lower leg shaft or prosthesis knee joint) of a prosthesis according to the present invention. The upward direction is proximal; downward is denoted as distal. “Ventral” is in FIG. 1 on the left; “dorsal” on the right.

The prosthetic foot 100 comprises a structural component 10 with a proximal connecting device 1 for fastening the prosthetic foot 100 to the lower leg shaft 200.

The structural component 10 defines or forms a forefoot section 9.

The proximal connecting device 1 is herein exemplarily designed as at least one joint. Alternatively, it optionally comprises at least one joint. The optional joint is herein exemplarily monoaxial or monocentric having one rotation axis which protrudes or projects into the drawing plane. Alternatively, the joint may be polycentric or polyaxial.

The structural component 10 exemplarily comprises a foot sole 2 or consists thereof.

The structural component 10 comprises an elastic element, herein exemplarily in the form of a ventrally arranged spring, herein exemplarily a leaf spring or bending spring.

The structural component 10 comprises a further elastic element, herein exemplarily in the form of a dorsally arranged bending spring 4.

According to the present invention, either the ventral or the dorsal elastic element is sufficient. However, the embodiment shown herein having two elements 3, 4 offers its particular advantages.

The elastic bending spring 3 is, in a first area 7a, connected to the structural component 10, herein to the foot sole 2. Alternatively, a plug-in solution is provided amongst others. In addition, it is advantageous if an elastic element like e.g. the ventrally arranged bending spring 3 or the dorsally arranged bending spring 4 is provided in a releasable manner thus being easily replaceable by the user. By said replaceability, the user may easily use elastic elements which appear to be optimal to the current requirement of the prosthesis (running, sport, walking, sightseeing, etc. due to their geometry or elasticity, spring effect or the like,).

According to the present invention, an adjusting device, by which the spring effect and/or the spring strength is adjustable for the prosthesis wearer, may be provided in several embodiments according to the present invention. The wearer may thus adapt the prosthesis according to his needs and activities temporarily or constantly.

Also the optionally provided elastic bending spring 4 is connected to the structural component 10, herein the foot sole 2, in a first area 7a. The same which herein applies to the bending spring 3 or its connection to the structural component 10 and/or to the lower leg shaft 200 may in several embodiments according to the present invention apply also to the bending spring 4.

The bending spring 3 ventrally contacts or abuts with a second area 7b of the bending spring 3 the lower leg shaft 200. It primarily serves for influencing the rolling dynamics of the prosthesis.

The bending spring 4 dorsally contacts with a second area 7b of the bending spring 4 the lower leg shaft 200. It primarily serves for influencing the damping or the dynamics of the prosthesis.

The second area 7b is optionally arranged to be shiftable relative to the lower leg shaft 200.

The second area 7b lies optionally in an end area of the elastic element, here the bending spring 3 or 4.

An optional intermediate element 8 may lie between the second area 7b and the lower leg shaft 200. The intermediate element 8 may be part of the elastic element, here the bending spring 3 or 4, or part of the lower leg shaft 200. The intermediate element 8 may be arranged to be shiftable relative to the second area 7b and/or to the lower leg shaft 200.

The intermediate element 8 may be designed as an adapter between the geometries of the second area 7b on the one hand and of the lower leg shaft 200 on the other.

An adjusting device which is not shown may be provided to define or fix the intermediate element 8 to the lower leg shaft 200 or to the elastic element in a predetermined or desired height of the lower leg shaft 200 or of the elastic element, here the bending spring 3 or 4.

The prosthetic foot 100 comprises an optional support element 5. With the latter, the bending spring 3 and the lower leg shaft 200 may be, e.g. directly or indirectly, connected to each other, for example in a form-fit and/or force-fit manner, preferably releasable adjustable. The supporting element 5 may advantageously render possible or contribute to the dispensability of the dorsal bending spring 3.

The supporting element 5 may optionally be shifted or adjusted by an optional height adjustment 6 (e.g. in the form of a plug-in opening and at least one plug-in pin) along the longitudinal direction of the lower leg shaft 200. Thus, the spring effect of the bending spring 3 may be advantageously adjusted.

The supporting element 5 may, in each case purely optionally, be available in each embodiment according to the present invention.

Optionally, the bending spring 3 and/or the bending spring 4 do not respectively contact (directly or indirectly) the lower leg shaft 200 between their first area 7a and second area 7b.

As can be seen in FIG. 1, the foot sole 2 of the prosthetic foot 100 of this embodiment optionally has no elastic element and/or no spring which would serve for its own elasticity. The space for mounting or constructing which is required in the prior art is advantageously not needed according to the present invention.

The structural component 10 is also not designed as a leaf spring by way of example and/or optionally does not comprise the same. This offers advantages in terms of manufacturing, fatigue strength and adjustability.

The first area 7a may be firmly connected to the structural component 10. It may be releasably connected thereto. In particular, the first area 7a may be connected to the structural component 10 by a latching, snapping, clamping or other kind of connection. The user of the prosthesis may thus deliberately disable or weaken the spring effect of the bending spring 3. This may be desired when sitting at a table; if the spring effect is disabled for sitting, this allows the prosthetic foot to go into a plantar extension, thus the prosthetic foot is on the ground like the healthy foot also. Optically, the prosthetic foot can no longer be distinguished from a healthy foot. The latching connection or another connection makes it possible, through or by a re-latching, which may be done before, during or after getting up from the chair, that the bending spring 3 is brought in position as a spring element with the a.m. effect.

The first area 7a may be connected to the structural component 10 in a predetermined angle. The angle may be changeable by a provided device or by a tool. The angle may be constant or unchangeable.

FIG. 2 shows in a purely exemplary embodiment of a prosthetic foot 100 according to the present invention in a second embodiment in lateral view, fastened to a lower leg shaft 200. The upward direction is denoted as towards proximal; downward as towards distal. “Ventral” lies like in FIG. 1 on the left; “dorsal” on the right. “Lateral” lies exemplarily behind the drawing plane; “medial” lies in front of it. These spatial indications or information apply as well to the embodiments shown in FIG. 3 and FIG. 4.

The proximal connecting device 1 is herein exemplarily designed as at least one joint, which is connected to an extension 11, e.g. being a tube or lower leg tube, thus ensuring the connection between the structural component 10 on the one hand and the lower leg shaft 200 on the other hand.

As indicated with double arrow in FIG. 2, the joint of the connecting device 1 may be connected to the structural component 10 via a shifting device and may be shifted or adjusted via the latter e.g. in the shown ventral-dorsal direction. Optionally, a shiftability in a lateral-medial direction is also or alternatively provided. This shiftability may be provided in each embodiment according to the present invention, e.g. also in that of FIG. 1.

As indicated with further double arrow in FIG. 2, the first area 7a may be connected via a shifting device to the structural component 10 and may be shifted or adjusted via the latter in e.g. the shown ventral-dorsal direction. A respective shifting or adjusting device is denoted with the reference numeral 12 in FIG. 2. This may be firmly fixed to the first area 7a or to the structural component 10, thus may either move or not when being shifted relative to the structural component 10. Optionally, a shiftability in a lateral-medial direction is likewise provided. This shiftability may in turn be provided in each embodiment according to the present invention, e.g. also in that of FIG. 1.

The shifting devices may be arranged to influence the spring effect of the bending spring 3, e.g. to increase it. The shifting devices may, therefore, serve as adjusting devices for the bending spring 3.

FIG. 3 shows in a purely exemplary embodiment a prosthetic foot 100 according to the present invention in a third embodiment in a lateral view, fastened to a lower leg shaft 200.

FIG. 3 shows the optional intermediate element 8, which is arranged between the lower leg shaft 200 and the second area 7b. The optional connector 13 may lie freely between the lower leg shaft 200 and the second area 7b, it may be form-fit connected to one or both components mentioned supra in a releasable or non-releasable manner.

The intermediate element 8 may be part of the connector 13, which may be designed as a rod or tube. The connector 13 may extend preferably from the structural component 10 to the lower leg shaft 200.

The intermediate element 8 may be understood as an intermediary or interlink between the lower leg shaft 200 and the bending spring 3. It may effect a, in particular geometrical, balance between the lower leg shaft 200 and the bending spring 3 in a lateral-medial direction. It may effect a balance between the curved surface of the lower leg shaft 200 and the optional straight surface of the bending spring 3. It may effect a protection of the lower leg shaft 200 against the mechanical load caused by the bending spring 3 being made, for example, of Carbon.

The intermediate element 8 may be understood as a guiding device for guiding the bending spring 3 at or along the lower leg shaft 200.

The optional connector 13 may be connected to the structural component 10, e.g. to its foot sole 2, in a further section 17 lying distally to the intermediate element 8.

As indicated in FIG. 3 with again a further double arrow, the further section 17 may also be connected to the structural component 10 via a shifting device, not shown, and may be shifted or adjusted by the latter in the indicated ventral-dorsal direction. Optionally, a shiftability in a lateral-medial direction is likewise provided. The shiftability may again be provided in each embodiment according to the present invention, e.g. also in that of FIG. 4.

The further section 17 may be mounted free of any spring or return effect, thus allowing a rotation or twisting of the connector 13 and/or of the second section 17 without having to overcome a spring effect needed thereto.

The connections between some components of the prosthesis according to the present invention which includes or consists of the prosthetic foot 100 and the lower leg shaft 200, may be such that the connections 1, 7a and/or 17 are mutually displaceable. In this, the respective distance between them, or preferably between some of them, is adjustable which may serve for the fitting or customization of the prosthesis to the user or to his/her activities.

FIG. 4 shows in a purely exemplary embodiment a prosthetic foot 100 according to the present invention in a fourth embodiment in a lateral view, fastened to a lower leg shaft 200.

FIG. 4 is based on FIG. 3. The exemplary embodiments shown in these two figures differ in that the optional connector 13 in FIG. 3 is connected to the structural component 10, e.g. its foot sole 2, via the section 17 on the ventral side of the connecting device 1, while the section 17 of FIG. 4 lies dorsally to the connecting device 1.

As can be seen in FIG. 4, the connector 13 extends, when seen from the side, from the ventral side of the lower leg shaft 200 (in the area of the second section 7b) to the dorsal side of the lower leg shaft 200, where it is connected to the foot sole 2 in section 17. In this, it may be concavely curved or both concavely and convexly. The latter design may be similar to a double curvature or an S-shape, as shown e.g. in FIG. 4.

An advantage associated with the particular shape of the connector 13 may be that when walking, there is an increasing spring effect with the increasing dorsal extension of the foot. The increasing spring effect results from the fact that the section 17, which may be configured or understood as a fulcrum, is arranged far dorsally in FIG. 4 in comparison with the embodiment of FIG. 3 and causes or effects the second section 7b moving towards the distal side (and correspondingly also back), as indicated by double arrow in FIG. 4. This may lead to an increased spring effect of the bending spring 3.

The intermediate element 8 may thus be designed as a height-shiftable or height-adjustable section with respect to the height of the bending spring 3.

It should be noted that also the bending spring 3 may have the course and shape of the connector 13. The advantages achieved with the connector 13 may thus be also achieved without the connector 13, but with a correspondingly designed and arranged bending spring 13.

In addition to (or instead of) the connector 13 shown in FIG. 4, which extends medially to the tube 11, it is also possible to provide a lateral connector, not shown in FIG. 4, which extends laterally to the tube 11. The connector which is not shown may for example be designed and connected like the connector 13.

FIG. 5a shows a prosthesis according to the present invention with a prosthetic foot 100 according to the present invention in a fifth embodiment in a lateral view, fastened to a lower leg shaft 200.

The illustration of FIG. 5a is based on FIG. 3 and FIG. 4. In FIG. 5a, a further embodiment of the intermediate element 8 and of the first area 7a is shown. For reason of simplicity and clarity, the optionally provided and arbitrarily designed connector 13 is not shown in FIG. 5a, even though it is or could be provided in an analogous arrangement to FIG. 4 in an exemplary embodiment.

The intermediate element 8 is designed as—with regard to the height (in y-direction) of the bending spring 3—height-shiftable or height-adjustable section. The bending spring 3 may move within the intermediate element 8 and relative thereto, at least in the y-direction.

In the exemplary illustration of FIG. 5a, the intermediate element 8 optionally comprises at least one or, like herein, two (or more) curved surfaces or sections. At least one of the curved surfaces or sections is arranged such that the bending spring 3, depending on the motion direction of the lower leg shaft 200 relative to the bending spring 3, is guided along the curved surfaces, contacts them and/or is limited by them, in particular in a x-direction being perpendicular to the height direction.

With a movement of the lower leg shaft 200, the bending spring 3 may contact the curved surfaces or sections (herein in short: surfaces) or move relative thereto, and thus move relative to the intermediate element 8.

The curved surfaces or sections of FIG. 5a, which are optional, may, like in FIG. 5a, respectively be convex towards the bending spring 3. Their (at least one) radius of curvature may each lie in the x, y-plane or in a plane parallel thereto.

In case of a relative movement, the movement depends in particular on the surface conditions of the curved surfaces and the bending spring 3 due to frictional movement and/or is limited thereby.

The direction of the movement of the lower leg shaft 200 is indicted by the double-sided arrow next to the illustrated x, y axes coordination system (above the bending spring, with regard to FIG. 5a). The movement along this double-sided arrow may be denoted as tilting movement. Due to the intermediate element 8 connected to the lower leg shaft 200, the leaf spring 3 is guided in this intermediate element 8 along the curved surfaces.

With the curved surfaces of the intermediate element 8, an exact and reliable guiding of the leaf spring 3 in the intermediate element 8 may be advantageously ensured. This may lead to a reliable and more stable guiding of the lower leg shaft 200, in particular relative to the prosthetic foot 100.

At the same time, the curved form of the surfaces of the intermediate element 8 which possibly contact the bending spring 3, allow a relatively large freedom of movement. Thus, the intermediate element 8 as designed in FIG. 5a and/or FIG. 5b may advantageously prevent or reduce an unintentional twisting of the bending spring 3 and thus an uneven or unplanned bending of the bending spring 3 effected or caused by the movement sequences of the prosthesis wearer.

To the left of the intermediate element 8 (with respect to FIG. 5a) a further, double-sided arrow with a y, z plane spanned perpendicularly to the drawing plane is schematically illustrated. This arrow indicates a further possible movement direction of the leaf spring 3 guided in the intermediate element 8. This movement direction is shown and described more closely in FIG. 5b.

Optionally, the first area 7a is designed to be shiftable or adjustable in x-direction. This movement direction is illustrated by the double-sided arrow below the first area 7a. Thus, the position of the spanning of the leaf spring 3 in or at the prosthetic foot 100 may be changed, for example, to achieve an individual adjustment for the prosthesis wearer. Such a shiftability, which possibly requires tools and/or may be releasably limited by a clamping mechanism, spanning mechanism, etc. may be made possible, for example, by the shifting or adjusting device 12, which is shown in FIG. 2 and which may, as shown in FIG. 7, be optionally designed as guiding elements 21 on which a slide 23, 29 (see FIG. 7) may drive or move.

The first area 7a is optionally designed with a further degree of freedom of movement as compared with the embodiment from FIG. 4. This is shown by the circular shape of the first area 7a in FIG. 5a. This further degree of freedom of movement is in particular a rotation about the x-axis. By this rotation about the x-axis, the leaf spring 3 may be moved or tilted in the y, z-plane (perpendicular to the plane of the drawing). This movement is described in detail in FIG. 5b.

The first area 7a may be designed as a joint. The joint may be a polyaxial and/or polycentric joint. The first area 7a may be a hinge, a saddle joint, a ball joint, etc.

The first area 7a may optionally have a joint effect, for instance for tilting, about the x-axis or with the x-axis as a rotation axis (or in a left-right direction in FIG. 5b); preferably, the first area has only this joint effect and no other one.

FIG. 5b shows the view of FIG. 5a rotated about 90 degrees to the right. In other words, the view of FIG. 5b is a view from the front onto the prosthesis according to the present invention with a prosthetic foot 100 according to the present invention of FIG. 5a.

In this view of FIG. 5b, the leaf spring 3 and the intermediate element 8 are seen in top view from the front. This top view corresponds to an illustration in the y, z-plane.

A possible movement direction of the prosthesis according to the present invention with the lower leg shaft 200 is illustrated by the double-sided arrow above the leaf spring and above the intermediate element 8. With this movement, the leaf spring 3 is guided again in the intermediate element 8 along (herein optionally two) optionally curved surfaces. This movement of the bending spring 3 in the y, z-plane may be referred to as tilting movement of the leaf spring 3.

The lower leg shaft 200, the tube 11, the intermediate element 8 and the leaf spring 3 may thus be rotated or tilted relative to the foot sole 2 about the first area 7a and the connecting device 1. With this movement possibility, which may be referred to as degrees of freedom of movement, the walking or running movement of the prosthesis wearer may be improved, in particular by improved weight shifting of the prosthesis wearer relative to the footprint or base plane of the foot sole 2 on a ground or floor.

The curved surfaces illustrated in FIG. 5b may be designed to be identical, similar or different in comparison to those illustrated in FIG. 5a. For example, the curved surfaces in FIG. 5b may be either more curved or less curved in comparison to the curved surfaces of FIG. 5a. This may lead respectively to a different movement behavior of the prosthesis wearer when walking.

The leaf spring 3 may be removable from the intermediate element 8 and/or from the first area 7a. This may be advantageously used, depending on the prosthesis wearer (size, weight) and possible movement behavior, in order to use different materials for the leaf spring 3 and/or different leaf spring strengths. Possible materials for the leaf spring 3 are plastics, glass-fiber reinforced plastics, carbon-fiber reinforced plastic, composite materials, metallic materials or combinations of these materials.

Depending on the prosthesis wearer, predetermined parameters of the position of the intermediate element 8 at the lower leg shaft 200, of the size and/or of the materials of the leaf spring 3 and/or other parameters may be selected.

The curved surfaces of the intermediate element 8, illustrated in FIG. 5a in the x, y-movement plane, and on the other side in the y, z-movement plane in FIG. 5b, may allow together with the lower leg shaft 200 according to the present invention and the prosthetic foot 100 according to the present invention a stable and safe walking of the prosthesis wearer. The movements may be superimposed in both motion planes, depending on the movement direction, anatomical conditions of the prosthesis wearer, etc. Such complex movement sequences may advantageously be stable and safely implemented by the illustrated intermediate element 8 with the respective curved surfaces.

The curved surfaces or sections of FIG. 5b, which are optional, may, as in FIG. 5b, each be convex towards the bending spring 3. Their (at least one) curve radius may respectively lie in the y, z-plane or in a plane parallel thereto.

The curved surfaces or sections of FIGS. 5a and 5b are, as described supra, each convexly curved towards the bending spring 3. In FIG. 5a, the curved surfaces or sections are each arranged on the right and on the left of the drawing plane (x, y-plane), this corresponds to respective planes in front and behind the drawing plane (y, z-plane) in FIG. 5b. In FIG. 5b, the curved surfaces or sections are each arranged on the right and on the left of the drawing plane (y, z-plane), respectively corresponds to respective planes in front of and behind the drawing plane (x, y-plane) in FIG. 5a

The curved surfaces or sections may likewise be differently arranged. For example, the curved surfaces or sections within the intermediate element 8 may each be convex towards the bending spring 3, wherein the curves may be designed three-dimensionally in the x, y, z-space. Thus, the bending spring 3 may in particular no longer flatly contact or abut against the curved surfaces or sections of the intermediate element 8, rather only in a punctiform manner. This contacting may be referred to as saddle-shaped abutment to the three-dimensional surface. The bending spring 3 may abut against the three-dimensional surface at a plurality of saddle-shaped points, which may be referred to as point-shaped regions.

The design and configuration of the three-dimensional surface with surfaces convexly curved towards the bending spring 3 may be designed differently and individually. This advantageously makes it possible to configure respectively different three-dimensional surfaces for individual prosthesis wearers and/or specific embodiments of a lower leg shaft 200 according to the present invention and/or specific embodiments of a prosthetic foot 100 according to the present invention.

Furthermore, different three-dimensional surfaces having different, in particular interchangeable, bending springs 3 may be combined and adjusted or adapted individually.

The frontal, although optional, limitation of the second area 7b in the intermediate element 8 is not shown in FIG. 5b nor in FIG. 6b for better illustration.

FIG. 6a shows the embodiment of FIG. 5a, however with a further embodiment of the intermediate element 8. In this embodiment, the leaf spring 3 is fixed in or at the intermediate element 8. The fixing may be a releasable or a non-releasable connection, e.g. a material-fit or form-fit connection. The leaf spring 3 may be clamped, glued or differently fixed in the intermediate element 8.

The movement of the leaf spring 3 with respect to the intermediate element 8 which is described above in FIGS. 5a and 5b based on the curved surfaces of the intermediate element 8 is realized in the embodiment of FIGS. 6a and 6b by a further connecting device 14. The further connecting device 14 may movably carry the intermediate element 8, and thus also the leaf spring 3, during a movement of the lower leg shaft 200. The intermediate element 8 with the leaf spring 3 may make or follow the movement of the lower leg shaft 200 in whole or in part, depending on the design of the further connecting device 14.

The further connecting device 14 may be a joint, for example a polyaxial and/or a polycentric joint, and/or a hinge.

The further connecting device 14 may be releasably or non-releasably connected to the lower leg shaft 200 and/or to the intermediate element 8.

The connecting device 14 may be designed to be shiftable relative to the lower leg shaft 200 and/or to the intermediate element 8.

Moreover, the description of FIGS. 5a and 5b, in particular with regard to the first area 7a, analogously applies to FIGS. 6a and 6b.

FIG. 7 shows a prosthetic foot 100 according to the present invention in a further embodiment.

The proximal connecting device 1 is shiftably connected by a slide to a shifting or adjusting device 12, herein optionally to guiding elements 21 of the shifting or adjusting device 12. The connection shown in the example of FIG. 7 between the proximal connecting device 1 and the shifting or adjusting device 12 is secured by screws 19 which ensure the position of the proximal connecting device 1 on the shifting or adjusting device 12. Instead of—one or more—screws 19, a different joint connection or fastening between the proximal connecting device 1 and the shifting or adjusting device 12 may be provided. This may be a clamp connection, click connection, plug connection or the like. Preferably, the connection may be designed to be released and closed without using tools.

The shifting or adjusting device 12 exemplarily comprises two guiding elements 21 (e.g. guiding rails), one or more guiding elements 21 are likewise provided by the present invention.

The proximal connecting device 1 comprises optionally the slide 23 shown in FIG. 7, which is engaged to and/or contacts the guiding elements 21 or is optionally connected thereto.

The shifting or adjusting device 12 is itself connected to the structural component or, as here, to the foot sole 2 via screws 25. It could be connected to the structural component or to the foot sole 2 with a different way, like e.g. welded, glued, etc.

In the particular design of FIG. 7, the shifting or adjusting device 12 serves for connecting also the first area 7a of the bending spring 3 (as an elastic element) to the foot sole 2.

The arrangement of the first area 7a and/or the proximal connecting device 1 on the shifting or adjusting device 12 allows not only to adjust the distance between the proximal connecting device 1 and the first area 7a which in turn allows an adaptation of the prosthetic foot 100 to the activity of the prosthesis wearer, the selected shoe form, the desired spring effect, etc. Such an arrangement allows the cost-effective manufacturing of the prosthetic foot 100 with only one shifting or adjusting device.

As can be seen in FIG. 7, the first area 7a of the bending spring 3 is received in a receptacle 27 (e.g. screwed or glued therein). The receptacle 27 which is limited by the bending spring 3 at both the front side of the bending spring 3 and at its rear side 3a, is connected to the shifting or adjusting device 12 by one, two (as shown in FIG. 7) or more slides 29. The slides 29 are engaged to and/or contact the guiding elements 21. Using the slide 29, the first area 7a may be shifted along the guiding elements 21. The connection between the slides 29 and the guiding elements 21 may optionally be designed like the connection between the slides 23 and the guiding elements 21.

The receptacle 27 for the first area 7a of the bending spring 3 may optionally be two-piece and may touch the first area 7a from the front and from the back. In this, the front section 27a and the rear section 27b are closer to each other in a bottom area than in an upper area. This may be achieved by e.g. a rounding or chamfering of the upper area of the front section 27a and the rear section 27b. The bigger distance provided on top may grant or concede the bending spring 3 a tension-free bending movement and may help that the clamping length of the first area 7a in the receptacle 27 may be kept small and/or the durability of the bending spring 3 is advantageously increased.

The receptacle 27, or optionally the first area 7a directly, may be connected to the slide 29 by a rotating joint 31 as shown in FIG. 7, or may alternatively be directly connected to the shifting or adjusting device 12 or to the foot sole 2.

The rotating joint 31 advantageously allows a rotation or tilting movement of the bending spring 3 in a left-right direction of the foot sole 2.

The rotation axis of the rotating joint 31 may optionally be perpendicular to the rotation axis of the proximal connecting device 1 and/or parallel to a longitudinal direction of the foot sole 2.

The foot sole 2, as shown in FIG. 7, has optionally no joint that would connect single foot sole sections to each other. The particular design proposed herein, may supersede such a joint.

As shown in FIG. 7, the foot sole 2 may be directly or indirectly connected to the shoe sole 33, e.g. glued.

In the present invention, neither the proximal connecting device 1 nor the first area 7a must be shiftable and/or be connected via the shifting or adjusting device 12 to the structural component and/or the foot sole 2. Likewise, the proximal connecting device 1 on the one hand and the first area 7a on the other hand may, unlike in FIG. 7, be guided on separate shifting or adjusting devices.

The shifting or adjusting device 12 or the guiding elements 21 may be the elements mentioned supra, on which the bending spring 3 is already shiftably arranged. In this, the distance between the proximal connecting device 1 and the bending spring 3, in particular its first area 7a, may be varied simply. Alternatively, not only one but several shifting or adjusting devices 12 are provided, e.g. one shifting or adjusting device 12 for the bending spring 3, another one for the proximal connecting device 1.

### LIST OF REFERENCE NUMERALS

100 prosthetic foot
10 structural component
1 connecting device
2 foot sole
3 bending spring
3a rear side
4 bending spring
5 supporting element
6 height adjustment
7a first area
7b second area
8 intermediate element
9 forefoot section
11 tube, extension
12 shifting or adjusting device
13 connector
14 further connecting device
17 section
19 screws
21 guiding elements
23 slide
25 screws
27 receptacle
29 slide
31 pivoting or rotating joint
33 shoe sole
200 lower leg shaft
x x-direction, x-axis
y y-direction, y-axis
z z-direction, z-axis

1. A prosthetic foot with a structural component having proximal connecting devices for fastening the prosthetic foot to a lower leg tube, lower leg shaft or prosthetic knee joint, wherein the proximal connecting device is designed as joint or comprises at least one joint, wherein the prosthetic foot comprises at least one elastic element or is connected thereto, wherein the elastic element is, in a first area thereof, connected to the structural component, in particular releasably, and wherein the elastic element comprises a second area for contacting the lower leg tube, lower leg shaft or prosthetic knee joint, in particular at a side, front side or rear side of the lower leg tube, lower leg shaft or prosthetic knee joint.

2. The prosthetic foot according to claim 1, wherein the structural component is, comprises or is connected to a foot sole.

3. The prosthetic foot according to claim 1, wherein the elastic element is, comprises or is connected to a leaf spring or bending spring.

4. The prosthetic foot according to claim 1, wherein the elastic element dorsally or ventrally rests on or abuts the lower leg tube, lower leg shaft or prosthetic knee joint.

5. The prosthetic foot according to claim 1, wherein an adjustable element is provided for releasably fastening a section of the elastic element to the lower leg tube, lower leg shaft or prosthetic knee joint.

6. The prosthetic foot according to claim 1, wherein the elastic element does not contact or touch the lower leg tube, lower leg shaft or prosthetic knee joint between the first area and the second area.

7. The prosthetic foot according to claim 1, with an adjusting device for varying or amending the height of the second area above the foot sole or for varying the distance between the first area and the second area.

8. The prosthetic foot according to claim 1, wherein the joint of the connecting device is connected to the structural component via a shifting device.

9. The prosthetic foot according to claim 1, wherein the first area is connected to the structural component via a shifting device.

10. The prosthetic foot according to claim 1, wherein the prosthesis or the prosthetic foot comprises a connector which, with at least an intermediate element thereof, is arranged between the lower leg shaft and the second area.

11. The prosthetic foot according to claim 10, wherein the connector is connected, in a further section lying distally to the intermediate element, to the structural component, in particular to the foot sole of said structural component.

12. The prosthetic foot according to claim 10, wherein the connector extends from the ventral side of the lower leg shaft in the area of the second section to the dorsal side of the lower leg shaft, where it is connected, in the section, to the foot sole.

13. A prothesis with a prosthetic foot according to claim 1, connected to a lower leg tube, lower leg shaft or prosthesis knee joint of the prosthesis.

14. The prosthetic foot according to claim 4, wherein the elastic element does not rest on or abut medially or laterally the lower leg tube, lower leg shaft or prosthetic knee joint.
